(19) United States
(12) Patent Application Publication
(10) Pub. No.: US 2007/0270942 A1
(43) Pub. Date: Nov. 22, 2007

(54) Galvanic Corrosion Methods and Devices for Fixation of Stent Grafts

(75) Inventor: Richard Thomas, Cloverdale, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/419,251

(22) Filed: May 19, 2006

(51) Int. Cl. A61F 2/06

(52) U.S. Cl. 623/1.46

(57) ABSTRACT

Methods and devices are provided to contribute to improved stent graft fixation within vessels at treatment sites. Improved stent graft fixation within vessels at treatment sites is provided by providing stent grafts and methods of making and using stent grafts having structural scaffoldings which undergo controlled galvanic corrosion in situ. Other embodiments include stent grafts having galvanic cells attached to the vessel luminal wall-contacting sides. Still other embodiments include stent grafts that undergo controlled galvanic corrosion and include at least one additional cell growth promoting factor.

### FIELD OF THE INVENTION

Methods and devices for preventing stent graft migration and endoleak using controlled pro-inflammatory galvanic corrosion in association with a stent grafts.

### BACKGROUND OF THE INVENTION

Stent grafts have been developed to treat abnormalities of the vascular system. Stent grafts are primarily used to treat aneurysms of the vascular system and have also emerged as a treatment for a related condition, acute blunt aortic injury, where trauma causes damage to an artery.

Aneurysms arise when a thinning, weakening section of a vessel wall dilates and balloons out. Aortic aneurysms (both abdominal and thoracic) are treated when the vessel wall expands to more than 150% of its normal diameter. These dilated and weakened sections of vessel walls can burst, causing an estimated 32,000 deaths in the United States each year. Additionally, aneurysm deaths are suspected of being underreported because sudden unexplained deaths, about 450,000 in the United States alone, are often simply misdiagnosed as heart attacks or strokes while many of them may be due to aneurysms.

U.S. surgeons treat approximately 50,000 abdominal aortic aneurysms each year, typically by replacing the abnormal section of vessel with a plastic or fabric graft in an open surgical procedure. A less-invasive procedure that has more recently been used is the placement of a stent graft at the aneurysm site. Stent grafts are tubular devices that span the aneurysm site to provide support without replacing a section of the vessel. The stent graft, when placed within a vessel at an aneurysm site, acts as a barrier between blood flow and the weakened wall of a vessel, thereby decreasing pressure on the damaged portion of the vessel. This less invasive approach to treat aneurysms decreases the morbidity seen with conventional aneurysm repair. Additionally, patients whose multiple medical comorbidities make them excessively high risk for conventional aneurysm repair are candidates for stent grafting.

While stent grafts represent improvements over previously-used vessel treatment options, there are still risks associated with their use. The most common of these risks is migration of the stent graft due to hemodynamic forces within the vessel. Stent graft migrations can lead to endoleaks, a leaking of blood into the aneurysm sac between the outer surface of the graft and the inner lumen of the blood vessel which can increase the risk of vessel rupture. Such migrations of stent grafts are especially possible in curved portions of vessels where hemodynamic forces are asymmetrical placing uneven forces on the stent graft. Additionally, the asymmetrical hemodynamic forces can cause remodeling of an aneurysm sac which leads to increased risk of aneurysm rupture and increased endoleaks.

Based on the foregoing, one goal of treating aneurysms is to provide stent grafts that do not migrate. To achieve this goal, stent grafts with stainless steel anchoring barbs and hooks that engage the vessel wall have been developed. Additionally, endostaples that fix stent grafts more readily to the vessel wall have been developed. While these physical anchoring devices have proven to be effective in some patients, they have not sufficiently ameliorated stent graft migration associated with current treatment methods in all cases.

An additional way to reduce the risk of stent graft migration is to administer to the treatment site, either before, during or relatively soon after implantation, a cell growth promoting factor (also known in some instances as an endothelialization factor). This administration can be beneficial because, normally, the endothelial cells that make up the portion of the vessel to be treated are quiescent at the time of stent graft implantation and do not multiply. As a result, the stent graft rests against a quiescent endothelial cell layer. If cell growth promoting factors are administered immediately before, during or relatively soon after stent graft deployment and implantation, the normally quiescent endothelial cells lining the vessel wall, and in intimate contact with the stent graft, will be stimulated to proliferate. The same will occur with smooth muscle cells and fibroblasts found within the vessel wall. As these cells proliferate they can grow around the stent graft such that the device becomes physically attached to the vessel wall rather than merely resting against it. Most stent grafts of this type provide cell growth promoting factors on the fabric of the stent graft. Because stent graft fabric is smooth, however, this area of the graft may not provide the optimal surface to promote cell growth.

Another method used to endothelialization and stent graft attachment is described in U.S. Pat. Nos. 5,871,536 and 6,165,214 issued to Lazarus (hereinafter the Lazarus patents). The Lazarus patents describe intraluminal vascular grafts made from biocompatible materials such as polyester (Dacron®) or polytetrafluoro-ethylene (PTFE) (Teflon®). Fixed attachment of the Lazarus vascular grafts to the vessel intima is provided by inducement of an inflammatory response between the outer surface of the intraluminal vascular graft and the inner wall of the vessel. The inflammatory response is caused by placing along the frame and/or tube structure a material known to cause an inflammatory response in tissues such as cat gut, nylon, cellulose, polylactic acids, polyglycolic acids or polyamino acids. However, coating a hydrophobic polymer such as PTFE or polyesters with the pro-inflammatory polymers is difficult and the resulting coatings are often unstable and prone to delaminate and separate form the stent graft surface. This posses a significant thrombotic risk to the patient and may result in graft failure due to incomplete or partial endothelialization.

Therefore, there remains a need for minimally invasive methods and materials that reduce stent graft-associated aneurysm rupture, endoleaks and stent graft migration.

### SUMMARY OF THE INVENTION

Embodiments according to the present invention include methods and devices that are useful in reducing the risk of implantable stent graft migration. More specifically, methods and devices that promote implantable stent graft attachment to blood vessel luminal walls are provided. One embodiment provides methods and devices useful for minimizing post-implantation stent graft migration following deployment at an aneurysmal treatment site and is also useful in preventing or minimizing post-implantation endoleak following stent-graft deployment at an aneurysmal treatment site.

Embodiments according to the present invention offer these advantages by providing pro-inflammatory metal portions of stent grafts thus promoting more secure anchoring of the stent graft. Specifically, in one embodiment, a stent graft is provided comprising one or more exposed bare metal portions having a coating of a dissimilar metal such that controlled galvanic corrosion is induced in situ resulting in a pro-inflammatory response. In one embodiment, at least one of the bare metal portions having dissimilar metal coating is found at the end of the stent graft.

In one embodiment of present invention comprise the stent graft comprises of a radically expandable structural member comprised of a first metal having a coating comprised of a second metal wherein the combination of the first metal and the second metal results in galvanic corrosion in situ. The first metal being selected from the group consisting of stainless steels, cobalt-chromium alloys, titanium alloys, nickel-titanium alloys, tantalum, titanium, Elgiloy®, and combinations thereof. The second metal being selected from the group consisting of gold, platinum, silver, iron, zinc, magnesium, zirconium and combinations thereof.

In another embodiment of the present invention the metallic radically expandable structural member is partially coated with a first and second dissimilar metal such that only the distal and proximal ends of the stent graft undergo in situ galvanic corrosion.

In another embodiment of the present invention the metallic radically expandable structural member is partially coated with a dissimilar metal such that only the distal end of the stent graft undergoes in situ galvanic corrosion.

In another embodiment of the present invention the metallic radically expandable structural member is partially coated with a dissimilar metal such that only the proximal end(s) of the stent graft undergoes in situ galvanic corrosion.

In another embodiment of the present invention the entire metallic radically expandable structural member is coated with a dissimilar metal such that the entire stent graft undergoes in situ galvanic corrosion.

In another embodiment of the present invention the stent graft is provided with galvanic cells attached to the exterior (luminal wall contacting side) of the stent graft such that only the galvanic cells undergo in situ galvanic corrosion.

The present invention also comprises methods. One method according to the present invention comprises a method for treating an aneurysm comprising providing a stent graft comprising one or more exposed bare metal portions having a coating of a dissimilar metal such that galvanic corrosion is induced in situ resulting in a pro-inflammatory response that promotes cell growth. In one embodiment, at least one of the bare metal portions having dissimilar metal coating is found at the end of the stent graft.

### BRIEF DESCRIPTION OF THE DRAWINGS

FIG. 1 depicts a stent graft made in accordance with the teachings of the present invention using a shape-memory metal such as nitinol (a nickel-titanium alloy) as the base material as the first metal and a second dissimilar metal coating the entire stent scaffolding.

FIG. 2 depicts another embodiment of the present invention wherein the nitinol stent graft scaffolding is partially coated with a second dissimilar metal.

FIG. 3 depicts a stent graft made in accordance with the teachings of the present invention wherein only the distal and proximal ends are coated.

FIG. 4 depicts a stent graft made in accordance with the teachings of the present invention having galvanic cells attached to the vessel luminal-contacting wall.

FIG. 5 depicts a schematic diagram of a representative stent graft that can be used in accordance with the present invention deployed at a treatment site.

FIG. 6 depicts a distal end of an injection and delivery catheter that can be used in accordance with the present invention.

FIG. 7 depicts a close-up view of the distal portion of a representative stent graft.

### DEFINITION OF TERMS

Prior to setting forth embodiments according to the present invention, it may be helpful to an understanding thereof to set forth definitions of certain terms that will be used hereinafter. Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. The term “comprises” means “includes.”

Aortic aneurysm: As used herein “aortic aneurysm” shall include a weak section of an animal's aorta. As used herein, an “aortic aneurysm” includes, without limitation, abdominal and thoracic aneurysms.

Base metal: As used herein the “base metal” is the metal (first metal) being coated with the second metal (coating metal). The base metal may act as either the anodic or cathodic metal depending on whether the second metal, or coating metal is more or less noble relative to the base metal.

Biocompatible: As used herein “biocompatible” refers to any material that does not cause injury or death to the animal or induce an adverse reaction in an animal when placed in intimate contact with the animal's tissues. Adverse reactions include, without limitation, inflammation, infection, fibrotic tissue formation, cell death, embolizations and/or thrombosis.

Bioactive Material (also referred to herein as a therapeutic agent): As used herein, “bioactive material(s)” shall include any, drug, compound, substance or composition that creates a physiological and/or biological effect in an animal. Non-limiting examples of bioactive materials include small molecules, peptides, proteins, hormones, DNA or RNA fragments, genes, cells, genetically-modified cells, cell growth promoting factors, matrix metalloproteinase inhibitors, autologous platelet gel, platelet rich plasma, either inactivated or activated, other natural and synthetic gels, such as, without limitation, alginates, collagens, and hyaluronic acid, polyethylene oxide, polyethylene glycol, and polyesters, as well as combinations of these bioactive materials.

Cell Growth Promoting Factors: As used herein, “cell growth promoting factors” or “cell growth promoting compositions” shall include any bioactive material having a growth promoting effect on vascular cells. Non-limiting examples of cell growth promoting factors include vascular endothelial growth factor (VEGF), platelet-derived growth factor (PDGF), platelet-derived epidermal growth factor (PDEGF), basic fibroblast growth factor (bFGF), acidic fibroblast growth factor (aFGF), transforming growth factor-beta (TGF-β), platelet-derived angiogenesis growth factor (PDAF) and autologous platelet gel (APG) including platelet rich plasma (PRP), platelet poor plasma (PPP) and thrombin.

Controlled Galvanic Corrosion: As used herein “controlled galvanic corrosion” refers to a process whereby the type and amount of dissimilar metals are regulated using skills know in the art to provide for a predetermined amount of galvanic corrosion sufficient to induce the desired amount of inflammation without completely compromising the stent scaffold's structural properties. “Predetermined” as used herein refers to determining to extent of galvanic corrosion that will occur in situ through a series of in vitro experiments designed to simulate in vivo physiological conditions. A desirable amount of galvanic corrosion is defined as sufficient corrosion to induce inflammation at the implantation site such that stent graft migration and endoleak is prevented. Determining the desired amount of galvanic corrosion such that such that stent graft migration and endoleak is prevented is accomplished using histopathology techniques and dissection on experimental animals post implantation as know to those skilled in the art.

Dissimilar Metal: As used herein “dissimilar metals” refers to metals that, when in physical contact with each other and exposed to an electrolytic medium such as saline, blood or other biological fluids, will undergo galvanic corrosion. The potential of a metal in a solution is related to the relative resistance to corrosion in a corrosive environment. Differences in corrosion potentials of dissimilar metals can be measured in specific environments by measuring the direction of the current that is generated by the galvanic action of these metals when immersed in a given environment. Such measurements could be repeated with all the possible combinations of metals in any corrosive solution. A non-limiting example of a dissimilar metal pair prone to galvanic corrosion in a saline environment is zinc and steel. In this environment, zinc is more electrochemically active than the steel such that when the two are physically connected, as on galvanized steel, the zinc coating corrodes while the steel does not. In this case, the zinc is the sacrificial metal while the steel is protected from corrosion. Current generated from the corrosion process flows from the zinc to the steel.

Endoleak: As used herein, “endoleak” refers to the presence of blood flow past the seal between an end of the stent graft and the vessel wall, and into the aneurysmal sac, when all such flow should be contained within its lumen.

Galvanic Corrosion: As used herein “galvanic corrosion” (also called ‘dissimilar metal corrosion’ or wrongly ‘electrolysis’) refers to corrosion damage induced when two dissimilar materials are coupled in a corrosive electrolyte (including blood, serum and other body fluids). It can occur when two (or more) dissimilar metals are brought into contact under physiological conditions.

Galvanic Corrosive Coating: As used herein “galvanic corrosive coating” refers to a combination of a base metal and a coating metal wherein the combination is conducive to in situ galvanic corrosion when placed in a physiological environment.

Implantable Medical Device: As used herein, “implantable medical device” includes, without limitation, stents and stent grafts used in the repair of vascular injuries.

In Situ: As used here in “in situ” refers to the stent graft situated in place at the treatment site. An in situ process is a process occurring in the patient's body under physiological conditions at the treatment site.

Migration: As used herein, “migration” refers to displacement of a stent or stent graft sufficient to be associated with a complication, for example, endoleak.

Noble Metal: As used herein a “Noble Metal” is the metal protected by the sacrificial metal in a dissimilar metal pair. All metals dissolve to some extent when they are wetted with a corrosive liquid. The degree of dissolution is greatest with active or sacrificial metals such as magnesium and zinc and they have the most negative potential. In contrast, noble or passive metals such as gold or platinum are relatively inert and have a more positive potential. Stainless steel is in the middle although it is more noble than carbon steel. The potential can be measured with a reference electrode and used to construct a galvanic series (ASTM Standard G82).

Passivity: As used herein “passivity” refers to a condition in which a piece of metal, because of an impervious covering of oxide or other compound, has a potential much more positive than that of the metal in the active state. The more positive a metal is the more noble it is and thus more resistant to galvanic corrosion.

Stent graft: As used herein “stent graft” shall include a fabric (or fabric and metal composite, and/or derivations and combinations of these materials) tube that reinforces a weakened portion of a vessel (in one instance, an aneurysm).

Treatment Site and Administration Site: As used herein, the phrases “treatment site” and “administration site” includes a portion of a vessel having a stent or a stent graft positioned in its vicinity. A treatment site can be, without limitation, an aneurysm site, the site of an acute traumatic aortic injury, the site of vessel narrowing or other vascular-associated pathology.

### DETAILED DESCRIPTION OF THE INVENTION

Embodiments according to the present invention include methods and devices that are useful in reducing the risk of implantable stent graft migration. More specifically, methods and devices that promote implantable stent graft attachment to blood vessel luminal walls are provided. One embodiment provides pro-inflammatory stent grafts having structural scaffoldings comprised of dissimilar metals useful for minimizing post-implantation stent graft migration following deployment at an aneurysmal treatment site and is also useful in preventing or minimizing post-implantation endoleak following stent-graft deployment at an aneurysmal treatment site.

As discussed above, an aneurysm is a swelling or expansion of a vessel lumen at a defined point and is generally associated with a vessel wall defect. Aneurysms are often multi-factorial asymptomatic vessel diseases that if left unchecked can result in spontaneous rupture, often with fatal consequences. One method to treat aneurysms involves a highly invasive surgical procedure where the affected vessel region is removed and replaced with a synthetic graft that is sutured in place. However, this procedure is extremely risky and generally only employed in otherwise healthy vigorous patients who can be expected to survive the associated surgical trauma. Elderly and feeble patients are not candidates for these aneurysmal surgeries, and, before the development of stent grafts, remained untreated and at continued risk for sudden death.

In contrast to the described invasive surgical procedures, stent grafts can be deployed with a cut down procedure or percutaneously using minimally invasive procedures. Essentially, a catheter having a stent graft compressed and fitted into the catheter's distal tip is advanced through an artery to the aneurysmal site. The stent graft is then deployed within the vessel lumen juxtaposed to the weakened vessel wall forming an inner liner that insulates the aneurysm from the body's hemodynamic forces thereby reducing the risk of rupture. The size and shape of the stent graft is matched to the treatment site's lumen diameter and aneurysm length. Moreover, branched grafts are commonly used to treat abdominal aortic aneurysms that are located near the iliac branch.

Stent grafts generally comprise a metal scaffolding having a biocompatible covering such a Dacron® (E.I. du Pont de Nemours & Company, Wilmington, Del.) or a fabric-like material woven from a variety of biocompatible polymer fibers. Other embodiments include extruded sheaths and coverings. The scaffolding is generally on the luminal wall-contacting surface of the stent graft and directly contacts the vessel lumen. The sheath material is stitched, glued or molded onto the scaffold. In other embodiments, the scaffolding can be on the graft's blood flow contacting surface or interior. When a self-expanding stent graft is deployed from the delivery catheter, the scaffolding expands to fill the lumen and exerts circumferential force against the lumen wall. This circumferential force is generally sufficient to keep the stent-graft from migrating and thus preventing endoleak. However, stent migration and endoleak can occur in vessels that have irregular shapes or are shaped such that they exacerbate hemodynamic forces within the lumen. Stent migration refers to a stent graft moving from the original deployment site, usually in the direction of the blood flow. Endoleak (as used herein) refers specifically to the seepage of blood around the stent ends to pressurize the aneurysmal sac or between the stent graft and the lumen wall. Stent graft migration can result in the aneurysmal sac being exposed to blood pressure again and increasing the risk of rupture. Endoleaks occur in a small percentage of aneurysms treated with stent grafts. Therefore, it would be desirable to have devices, compositions and methods that minimize post implantation stent graft migration and endoleak.

Tissue in-growth and endothelialization around the stent graft have been proposed as methods to reduce the risk of stent graft migration and endoleak. Certain embodiments according to the present invention provide mechanisms to further stimulate tissue in-growth at one or more portions of a stent graft by providing a stent graft with one or more bare metal portions comprised of dissimilar metals that undergo galvanic corrosion in situ. Without wishing to be bound by this theory, the present inventor postulates that when an implanted metallic medical device undergoes galvanic corrosion in situ reactive metal ions, hydrogen ions, reactive oxygen species, and reactive nitrogen species into the surrounding tissues resulting in an inflammatory response (thus the in situ galvanic corrosion process is referred to herein as “pre-inflammatory”). This theory is supported by the observations that combinations of nitinol (NiTi) with platinum iridium (PtIr) and gold palladium (AuPd) alloys used for radiopaque markers result in high corrosion rates in situ (see, for example, R. Venugolapan and C. Trepanier, Assessing the corrosion behavior of Nitinol for minimally-invasive device design, Minimally Invasive Therapy & Allied Technologies. 9:67-75 (2000), incorporated herein by reference for all that it teaches related to in situ of metallic medical devices). The inflammation caused by the pro-inflammatory galvanic corrosion of the present invention results in activation of the innate immune system at the treatment site. This process can result in the recruitment of specific immune cells, and inflammation that may seal the stent graft to the vessel lumen preventing endoleak. Moreover, the recruitment of activated immune cells may result in chemokine and cytokine responses that including cell growth factors that promote healing and graft endotheializatrion.

Traditionally, the medical device community has gone to great lengths to avoid in situ galvanic corrosion (see, for example, S. Shabalovskaya, Surface corrosion and biocompatibility of Nitinol as an implant material, Bio-medical Materials and Engineering 12:69-109 (2002) incorporated herein by reference for all that it teaches related to in situ of metallic medical devices). The fundamental criterion for choosing metallic implant materials has been biocompatibility, required mechanical strength and reasonable corrosion resistance. Metallic implants are generally made from one of three material types: austenitic stainless steels (chromium-nickel stainless steels commonly known as 18-8 or 300 series), cobalt-chromium allows, and titanium and its alloys. These materials are acceptable in the physiological environment due to their passive and inert oxide surface layer. The alloying elements generally have a specific physiological role and thus are well tolerated in trace amounts. Cobalt-chromium alloys have excellent corrosion resistance but poor frictional properties and thus are commonly used to fabricate vascular stents abut are generally avoided as joint prostheses.

Corrosion is one of the major problems associated with metals and alloys used for implantable medical devices. Consequently, significant efforts have been brought to bear by the medical device engineering community to minimize this problem. Corrosion of implants in the aqueous medium of body fluids takes place via electrochemical reactions. The electrochemical reactions that occur on a medical devices surface are identical to the reactions that take place if the same metal was immersed in sea water. The metallic components of the alloy are oxidized to their ionic forms and the dissolved oxygen is reduced to hydroxyl ions. Thus the metals and alloys used in surgical implants are generally provided with a protective coating such as a polymer or passivity. (See generally, U. Kamachi Mudali, T. M. Sridhar and Baldev Raj, Corrosion of Bio Implants, Sadhana Vol. 28, parts 23 and 4 June/August 2003, pp. 601-637, incorporated herein by reference for all that it teaches related to in situ of metallic medical devices).

There are many types of corrosion known to affect metallic medical devices including pitting corrosion, crevice corrosion, fretting corrosion and galvanic corrosion. All types of corrosion can result in the release of pro-inflammatory chemical species into surrounding tissues and increase structural fatigue and eventual device failure. Thus, as stated, corrosion prevention has long been a significant focus of effort in the medical device industry. Of the aforementioned corrosion types, galvanic corrosion is of particular interest to the present inventor because it can be controlled and modulated resulting in a medical device that can be tuned to release specific amounts of pro-inflammatory compounds without compromising (entirely) the medical device's structural and mechanical properties.

For galvanic or dissimilar or electrolytic corrosion to occur, three conditions must be met: the metal joint must be wet with a conductive liquid; there must be metal to metal contact and the metals must have sufficiently different potentials. In the present invention the conductive liquid or electrolyte is a physiological fluid such as blood or blood plasma. Galvanic corrosion can only occur if the dissimilar metals are in electrical contact. The contact may be direct or by an external attachment such as a metal suture. All metals dissolve to some extent when they are wetted with a conductive liquid. The degree of dissolution is greatest with active or sacrificial metals such as magnesium and zinc and they have the most negative potential. In contrast, noble or passive metals such as gold or platinum are relatively inert and have a more positive potential. Stainless steel is in the middle although it is more noble than carbon steel. When two connected metals are in contact with a conducting liquid, the more active metal will corrode and protect the noble metal. Zinc is more negative than steel and so the zinc coating of galvanized steel will corrode to protect the steel at scratches or cut edges. The stainless steels, including austenitic stainless steels (chromium-nickel stainless steels commonly known as 18-8 or 300 series), are more positive than zinc and steel, so when stainless steel is in contact with galvanized steel and is wet, the zinc will corrode first, followed by the steel, while the stainless steel will be protected by this galvanic activity and will not corrode. The rate of galvanic attack is governed by the size of the potential difference.

Table 1 presents one representation of the galvanic series (which may change slightly depending on the corrosive (conductive) properties of the surrounding environment). The left hand column provides a descending list of sacrificial anionic metals. The higher the metal or alloy is on the list, the greater it's negative potential and thus the better sacrificial member of a dissimilar pair it makes. The right hand column depicts the noble metals. The higher the metal or alloy is on the list the less noble it is. Thus, by closely matching the anionic metal to the noble metal the extent of galvanic corrosion can be controlled.

TABLE 1
The Galvanic Series.

| Anodic or Least Noble | Cathodic or Most Noble |
|---|---|
| magnesium | manganese bronze (ca 675), tin |
| magnesium alloys | bronze |
| zinc | (ca903, 905) |
| aluminum 5052, 3004, 3003, 1100, | silicone bronze |
| 6053 | nickel silver |
| cadmium | copper - nickel alloy 90-10 |
| aluminum 2117, 2017, 2024 | copper - nickel alloy 80-20 |
| mild steel (1018), wrought iron | 430 stainless steel |
| cast iron, low alloy high strength steel | nickel, aluminum, bronze |
| chrome iron (active) | (ca 630, 632) |
| stainless steel, 430 series (active) | monel 400, k500 |
| 302, 303, 321, 347, 410, 416, stainless | silver solder |
| steel (active) | nickel (passive) |
| ni - resist | 60 ni-15 cr (passive) |
| 316, 317, stainless steel (active) | inconel 600 (passive) |
| carpenter 20cb-3 stainless (active) | 80 ni-20 cr (passive) |
| aluminum bronze (Ca 687) | chrome iron (passive) |
| hastelloy c (active) inconel 625 (active), | 302, 303, 304, 321, 347, |
| titanium (active) | stainless steel |
| lead - tin solders | (passive) |
| lead | 316, 317, stainless steel |
| tin | (passive) |
| inconel 600 (active) | carpenter 20 cb-3 stainless |
| nickel (active) | (passive), |
| 60 ni-15 cr (active) | incoloy 825 nickel - |
| 80 ni-20 cr (active) | molybdeum - |
| hastelloy b (active) | chromium - |
| brasses | iron alloy (passive) |
| copper (ca102) | silver |
| | titanium (pass.) Hastelloy c & |
| | c276 |
| | (passive), |
| | inconel 625 (pass.) |
| | Graphite |
| | zirconium |
| | gold |
| | platinum |

In the present invention the implantable medical devices, specifically stent grafts, comprise a structural member, or scaffolding, made of a base metal that provides the mechanical and structural properties to the stent graft. Non-limiting examples of suitable base metals include stainless steel, cobalt-chromium alloys, nickel-titanium alloys, tantalum, titanium, Elgiloy® (Elgiloy® is a registered trademark of Elgin National Watch Company Corporation Illinois, 107 national St. Elgin, Ill.) and the like. Elgiloy® comprises 15.5% nickel, 40% cobalt, 20% chromium, 7.0% molybdenum, 2% manganese, 0.15% carbon, 0.01% beryllium and the remainder being iron. In some embodiments of the present invention the base metal serves as the sacrificial metal, or anodic metal and is coated with a noble metal such as, but not limited to platinum, gold, zirconium and silver. The choice of noble metal will depend on the choice of base metal, which in turn depends on the mechanical and structural properties the stent graft engineer desires. Structural and mechanical properties of stent grafts and the corresponding base metals used to achieve these properties are well known to those having skill in the mechanical arts as illustrated by U.S. Pat. Nos. 5,907,893, 6,270,524 and 6,592,614; all of which are herein incorporated by reference for all they teach regarding stent graft design and construction. The stent grafts of the present invention may also be provided with a polymer fabric covering made form Dacron®, Teflon® or the like.

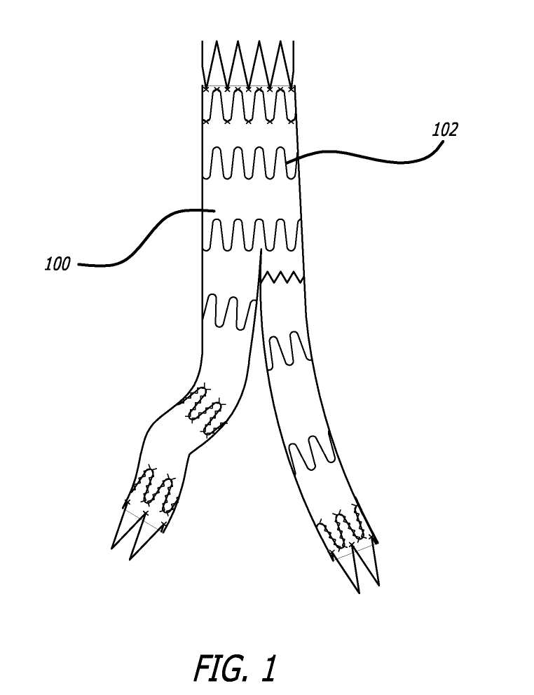

In one embodiment of the present invention a stent graft 100 as depicted in FIG. 1 is made using a shape-memory metal such as nitinol (a nickel-titanium alloy) as the base material. The stent grafts structural scaffolding is manufactured according to methods known in the art and sized to be useful in treating aortic abdominal aneurisms (AAA). The structural scaffolding 102 is then coated with gold using methods known to those skilled in the art such as plasma vapor deposition, sputtering, electroplating, dipping and the like. Suitable methods for coating metallic medical devices with noble metals such as gold are taught in U.S. Pat. No. 6,099,561; the entire contents of which are herein incorporated by references, (specifically those methods related to providing metallic medical devices with coating for noble metals).

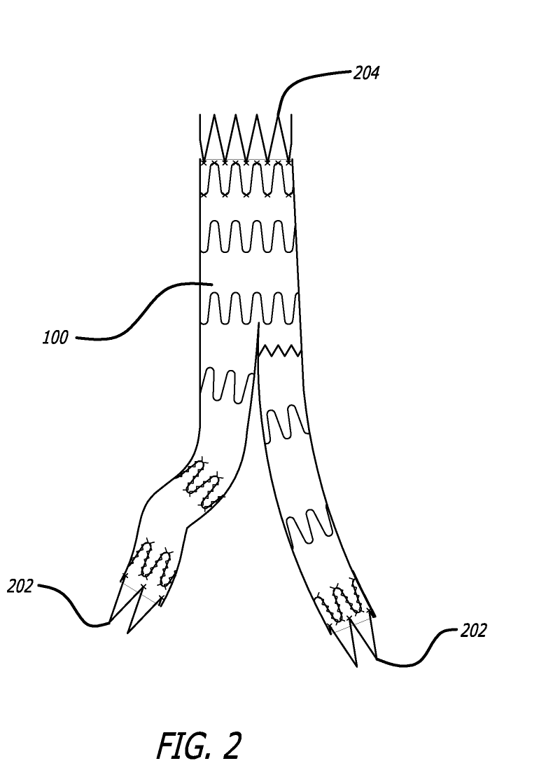

In another embodiment of the present invention the nitinol stent graft scaffolding is partially coated with a noble metal as depicted in FIG. 2. In one embodiment both the distal 202 and proximal ends 204 are coated. The coating 501 extending from approximately 0.1 mm up to and including approximately 5 cm from the ends (nested ranges included).

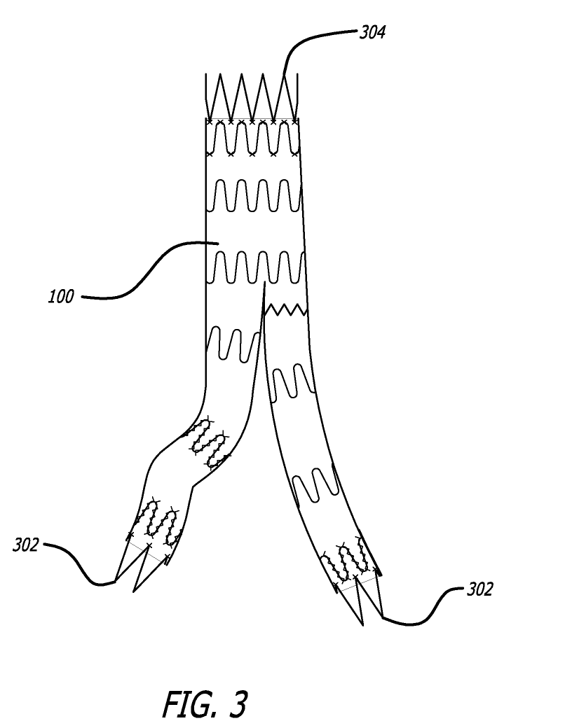

In another embodiment only the distal 302 or the proximal ends 304 are coated as depicted in FIG. 3. In an alternate embodiment only the stent graft's proximal end is coated. In another embodiment only the stent graft's proximal end is coated. As used herein distal refers to the end of the stent closest to the aortic bifurcation and proximal refers to the end closest the brain.

In yet an alternative embodiment of the present invention the base metal used to form the structural scaffolding of the vascular sent is cathodic and is coated with a less noble, or anodic sacrificial metal. For example, and not intended as a limitation, a stent graft is made using a shape-memory metal such as nitinol as the base material. The stent grafts structural scaffolding is manufactured according to methods known in the art and sized to be useful in treating AAA. The structural scaffolding is then coated with magnesium, zinc or iron using methods known to those skilled in the art such as plasma vapor deposition, sputtering, electroplating, dipping and the like. The coating of sacrificial metal may cover the entire stent surface or may be limited to one or more ends as described immediately above.

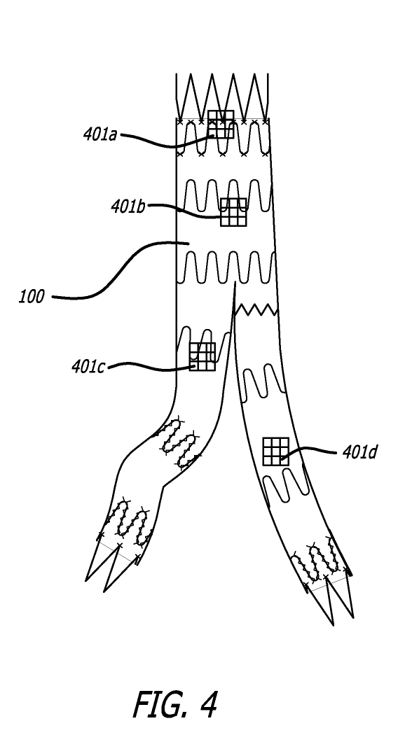

In another embodiment of the present invention the sent graft may comprise a structural scaffolding made from a biocompatible material such as but not limited to a metal or polymer. The stent graft may also be provided with a polymer fabric covering made form Dacron®, Teflon® or the like. In this embodiment of the present invention the stent graft is fitted with one or more galvanic cells located on the vessel luminal wall contacting side. A galvanic cell as used herein refers to a patch-like composition comprised a pair of dissimilar metals such that galvanic corrosion occurs in situ. FIG. 4 depicts a stent graft made in accordance with the teachings of the present invention having galvanic cells 401a-d affixed to the vessel luminal wall-contacting surface. The galvanic cells of the present invention can be affixed to the stent graft using any means known to those in the art including, without limitation sewing, weaving, molding, gluing, and stapling. In one embodiment of the present invention galvanic cells comprising stainless steel having a zinc coating is affixed to the vessel luminal wall-contacting surface using a biocompatible cyanoacrylate adhesive. In another embodiment of the present invention the galvanic cell comprises a base metal selected from the group consisting of stainless steel, cobalt-chromium alloys, nickel-titanium alloys, tantalum, titanium, Elgiloy®, and combinations thereof and the coating metal is selected from the group consisting of gold, platinum, silver, iron, zinc, magnesium, zirconium and combinations thereof.

In another embodiment of the present invention the stent graft having a galvanic corrosive coating is further provided with at least one a cell growth promoting factor on the one or more bare metal structural scaffolding portions. The cell growth factor (other than the galvanic coating itself) promotes growth of cells from the vascular endothelium around the bare metal portions. Other embodiments according to the present invention provide mechanisms to further stimulate tissue in-growth around a stent graft by providing a substance comprising a biocompatible polymer and a cell growth promoting factor on all or a subset of all bare metal portions found on a particular stent graft at a location other than the ends. In other embodiments, instead of or in addition to being found on bare metal portions of a stent graft, the substance comprising a biocompatible polymer and a cell growth promoting factor can be attached or woven into the material that forms the stent graft itself. As will be understood by one of skill in the art, however, and in light of further description provided herein, including the substance comprising a biocompatible polymer and a cell growth promoting factor on bare metal portions that can then be attached to the stent graft material can provide a more efficient manufacturing process than including the substance within the stent graft material itself. Both approaches, either alone or in combination, however, are included within the scope of the present invention.

Cell growth can be promoted by a variety of growth factors including, but not limited to vascular endothelial growth factor (VEGF), platelet-derived growth factor (PDGF), platelet-derived epidermal growth factor (PDEGF), fibroblast growth factors (FGFs) including acidic FGF (also known as FGF-1) and basic FGF (also known as FGF-2), transforming growth factor-beta (TGF-β), platelet-derived angiogenesis growth factor (PDAF). Cell growth can also be stimulated by induced angiogenesis, resulting in formation of new capillaries in the interstitial space and surface endothelialization, particularly by VEGF and acidic and basic fibroblast growth factors.

The discussion of these factors is for exemplary purposes only, as those of skill in the art will recognize that numerous other growth factors have the potential to induce cell-specific endothelialization and induce cell growth. Co-pending U.S. patent application Ser. No. 10/977,545, filed Oct. 28, 2004 which is hereby incorporated by reference, discloses injecting autologous platelet gel (APG) into the aneurysmal sac and/or between an implanted stent graft and the vessel wall to induce endothelialization of the stent graft to prevent stent graft migration and resulting endoleak. Autologous platelet gel is formed from autologous platelet rich plasma (PRP) mixed with thrombin and calcium. The PRP contains a high concentration of platelets that can aggregate for plugging, as well as release high levels of cytokines, growth factors or enzymes following activation by thrombin. The development of genetically-engineered growth factors also is anticipated to yield more potent endothelial cell-specific growth factors. Additionally it may be possible to identify small molecule drugs that can induce cell growth and/or endothelialization. Thus, the stent grafts according to the present invention can improve tissue in-growth through providing substances that promote cell growth near the ends of the stent graft, or at any other point along the length of the stent graft, and in some embodiments further by providing and releasing an endothelialization factor at one or more ends or along the length of the stent graft.

In one embodiment according to the present invention, cell growth promoting factors are delivered to a treatment site within a vessel lumen associated with a stent graft. The vessel wall's blood-contacting lumen surface comprises a layer of endothelial cells. In the normal mature vessel the endothelial cells are quiescent and do not multiply. Thus, a stent graft carefully placed against the vessel wall's blood-contacting luminal surface rests against a quiescent endothelial cell layer. However, if cell growth promoting compositions are present, the normally quiescent endothelial cells lining the vessel wall, and in intimate contact with the stent graft luminal wall contacting surface, will be stimulated to proliferate. The same will occur with smooth muscle cells and fibroblasts found within the vessel wall. As these cells proliferate they will grow into and around the stent graft lining such that the stent graft becomes physically attached to the vessel lumen rather than merely resting against it.

In one embodiment of the present invention, the cell growth promoting factors are coated, or paved, onto the bare metal portions of the stent graft in a polymeric material. The basic requirements for the polymeric material to be used in the stent grafts of the present invention are biocompatibility and the capacity to be chemically or physically reconfigured under conditions which can be achieved in vivo. Such reconfiguration conditions can involve heating, cooling, mechanical deformation, (e.g., stretching), or chemical reactions such as polymerization or cross-linking.

Suitable polymeric materials for use in the invention include both biodegradable and biostable polymers and copolymers of carboxylic acids such as glycolic acid and lactic acid, polyalkylsulfones, polycarbonate polymers and copolymers, polyhydroxybutyrates, polyhydroxyvalerates and their copolymers, polyurethanes, polyesters such as poly(ethylene terephthalate), polyamides such as nylons, polyacrylonitriles, polyphosphazenes, polylactones such as polycaprolactone, polyanhydrides such as poly[bis(p-carboxyphenoxy)propane anhydride] and other polymers or copolymers such as polyethylenes, hydrocarbon copolymers, polypropylenes, polyvinylchlorides and ethylene vinyl acetates.

In one embodiment according to the present invention, suitable biocompatible and biodegradable polymers include polyglycolic acid, poly˜glycolic acid/poly-L-lactic acid copolymers, polycaprolactive, polyhydroxybutyrate/hydroxyvalerate copolymers, poly-L-lactide, polydioxanone, polycarbonates, and polyanhydrides.

In one embodiment, the cell growth promoting stents grafts of the present invention utilize biodegradable polymers, with specific degradation characteristics to provide material having a sufficient lifespan for the particular application. As used herein, “biodegradable” is intended to describe polymers and copolymers that are non-permanent and removed by natural or imposed therapeutic biological and/or chemical processes. As such, bioerodable or bioabsorbable polymers and the like are intended to be included within the scope of that term.

The polymeric materials used in coating the cell growth promoting stent grafts of the present invention can additionally be combined with a variety of therapeutic agents for in situ delivery. Furthermore, the stent grafts having a galvanic corrosive coating without an additional growth promoting agent can also be provided with therapeutic agents for in situ delivery. Examples of such materials for use in coronary artery applications are anti-thrombotic agents, e.g., prostacyclin, heparin and salicylates, thrombolytic agents e.g. streptokinase, urokinase, tissue plasminogen activator (TPA) and anisoylated plasminogen-streptokinase activator complex (APSAC), vasodilating agents i.e. nitrates, calcium channel blocking drugs, anti-proliferative agents i.e. colchicine and alkylating agents, intercalating agents, antisense oligonucleotides, ribozymes, aptomers, growth modulating factors such as interleukins, transformation growth factor β and congeners of platelet derived growth factor, monoclonal antibodies directed against growth factors, anti-inflammatory agents, both steriodal and non-steroidal, modified extracellular matrix components or their receptors, lipid and cholesterol sequestrants and other agents which can modulate vessel tone, function, arteriosclerosis, and the healing response to vessel or organ injury post intervention. In applications where multiple polymer layers are used, different pharmacological agents could be used in different polymer layers.

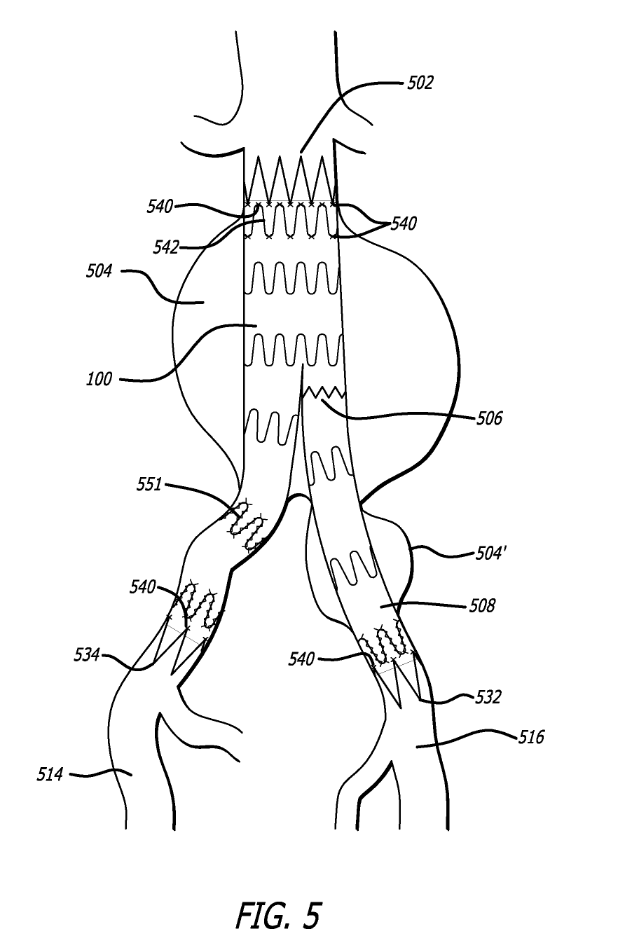

In one embodiment, a stent graft is provided “pre-loaded” into a delivery catheter. In an exemplary embodiment, a stent graft 100 is fully deployed to the site of an abdominal aortic aneurysm through the right iliac artery 514 to an aneurysm site 504 and 504' (FIG. 5). The stent graft 500 depicted in FIG. 5 has a distal end 502 comprised of bare metal portion and an iliac leg 508 also with a bare metal portion 532 to anchor the stent graft in the left iliac artery 516. Stent graft 500 is deployed first in a first delivery catheter and the iliac leg 508 is deployed in a second delivery catheter. The stent graft 500 and iliac leg 508 are joined with a 2 cm overlap of the two segments 506. In the embodiment depicted in FIG. 5, the bare metal portions 502, 532, 534 are found at the ends of the stent graft. These bare metal portions 502, 532, 534 are attached to the stent graft 100 at connection points 540 by any appropriate method including, without limitation, by stitching. Embodiments of the present invention can also comprise bare metal portions along the length of stent graft 100 such as those depicted by, for example, bare metal portions 542 and 551. In one embodiment, bare metal portions, such as that depicted by 542, can be provided for further structural support of stent graft 100 and for release of cell growth promoting factors. As will be understood by one of ordinary skill in the art, these bare metal portions can be found on any combination, number or position on a particular stent graft. One embodiment of bare metal portions 702 and 742, and connection points 740 of stent graft 100 can be seen in more detail in FIG. 7.

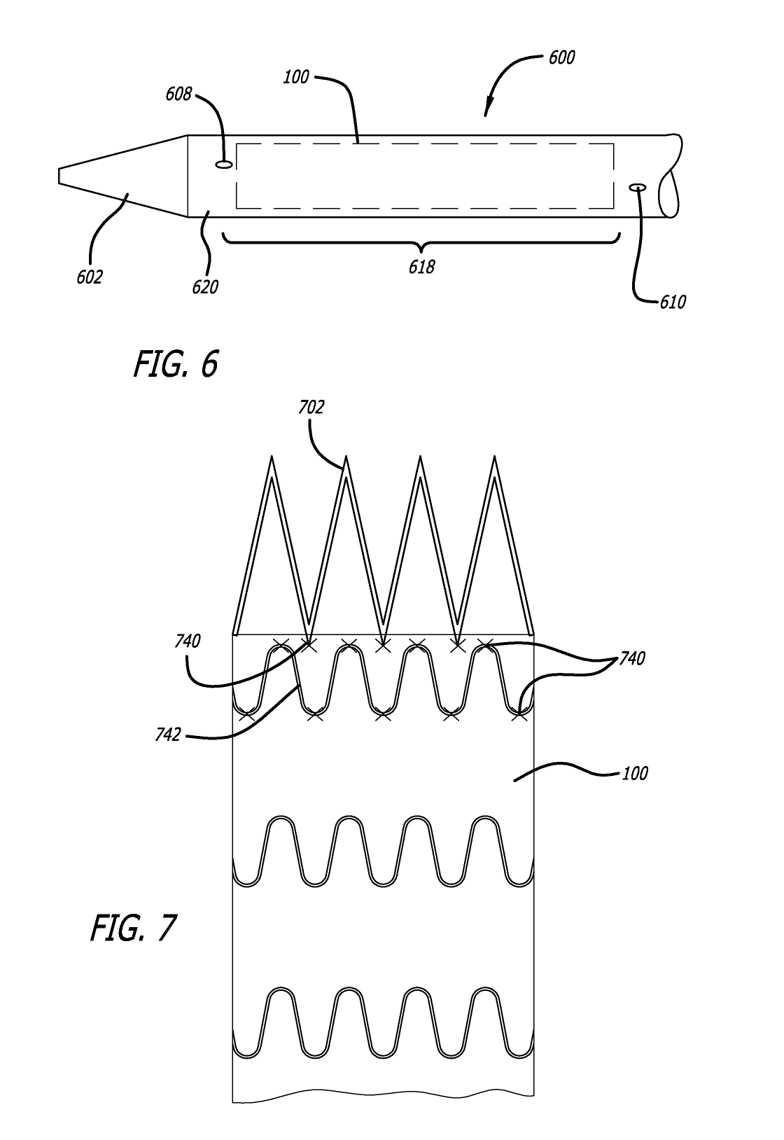

In another embodiment, a stent graft comprising a substance that promotes cell growth on one or more bare metal portions is pre-loaded into a delivery catheter such as that depicted in FIG. 6. Stent graft 100 is radially compressed to fill the stent graft chamber 618 in the distal end 602 of delivery catheter 600. The stent graft 600 is covered with a retractable sheath 620. Catheter 600 has two injection ports 608 and 610 for delivering the biocompatible polymer and cell growth promoting factor to the compressed stent graft. In this embodiment, the coating material is injected through either or both of injection ports 608 and 610 to wet stent graft 100. Stent graft 100 is then deployed to the treatment site as depicted in FIG. 5.

The field of medical device coatings is well established and methods for coating stent grafts with drugs, with or without added polymers, are well known to those of skill in the art. Non-limiting examples of coating procedures include spraying, dipping, waterfall application, heat annealing, etc. The amount of coating applied to a stent graft can vary depending upon the desired effect of the compositions contained within the coating. The coating can be applied to the entire stent graft or to a portion of the stent graft. Thus, various drug coatings applied to stent grafts are within the scope of embodiments according to the present invention.

Unless otherwise indicated, all numbers expressing quantities of ingredients, properties such as molecular weight, reaction conditions, and so forth used in the specification are to be understood as being modified in all instances by the term “about.”

Variations on embodiments will become apparent to those of ordinary skill in the art upon reading the foregoing description.

Furthermore, numerous references have been made to patents and printed publications throughout this specification. Each of the above cited references and printed publications are herein individually incorporated by reference in their entirety.

In closing, it is to be understood that the embodiments according to the invention disclosed herein are illustrative. Other modifications can be employed. Thus, by way of example, but not of limitation, alternative configurations invention can be utilized in accordance with the teachings herein.

I claim:

1. A stent graft comprising a structural scaffold comprising a first dissimilar metal coated with a second dissimilar metal wherein said stent graft undergoes controlled galvanic corrosion in situ.

2. The stent graft according to claim 1, wherein said first dissimilar metal is selected from the group consisting of stainless steels, cobalt-chromium allows, titanium alloys, nickel-titanium alloys, tantalum, titanium, Elgiloy®, and combinations thereof.

3. The stent graft according to claim 1 wherein said second dissimilar metal is selected from the group consisting of gold, platinum, silver, iron, zinc, magnesium, zirconium and combinations thereof.

4. A stent graft according comprising a structural scaffolding having at least one galvanic cell attached to the vessel luminal wall-contacting side attached thereto.

5. The stent graft according to either of claims 1 or 4 further comprising at least one substance that promotes cell growth.

6. The stent graft according to claim 5, wherein said cell growth promoting factor is basic fibroblast growth factor.

7. A stent graft comprising a structural scaffold comprised of a nickel-titanium alloy and a coating of a dissimilar metal comprised of gold said scaffolding undergoes controlled galvanic corrosion in situ.

8. A stent graft comprising a structural scaffold comprised of a nickel-titanium alloy and a coating of a dissimilar metal comprised of iron said coating undergoes controlled galvanic corrosion in situ.

9. A method for treating an aneurysm comprising:
providing to a patent in need thereof a stent graft comprising a structural scaffold comprising a first dissimilar metal coated with a second dissimilar metal wherein said stent graft undergoes controlled galvanic corrosion in situ.

10. The method according to claim 9 wherein said first dissimilar metal is selected from the group consisting of stainless steels, cobalt-chromium allows, titanium alloys, nickel-titanium alloys, tantalum, titanium, Elgiloy®, and combinations thereof.

11. The method according to claim 9 wherein said second dissimilar metal is selected from the group consisting of gold, platinum, silver, iron, zinc, magnesium, zirconium and combinations thereof.

12. A method for treating an aneurysm comprising:
providing to a patent in need thereof a stent graft comprising a structural scaffolding having at least one galvanic cell attached to the vessel luminal wall-contacting side attached thereto.

13. The method according to either of claims 9 or 12 wherein said method further comprises a stent-graft having at least one substance that promotes cell growth.

14. The method according to claim 13, wherein said cell growth promoting
factor is basic fibroblast growth factor.

15. A method for treating an aneurysm comprising providing to a patient in need thereof a stent graft comprising structural scaffold comprised of a nickel-titanium alloy and a coating of a dissimilar metal comprised of gold said scaffolding undergoes controlled galvanic corrosion in situ.

16. A method for treating an aneurysm comprising providing to a patient in need thereof stent graft comprising a structural scaffold comprised of a nickel-titanium alloy and a coating of a dissimilar metal comprised of iron said coating undergoes controlled galvanic corrosion in situ.
